(19) United States
(12) Patent Application Publication
(10) Pub. No.: US 2022/0409061 A1
(43) Pub. Date: Dec. 29, 2022

(54) FACE MASK WITH TEMPERATURE DISPLAY

(71) Applicant: UNITED ARAB EMIRATES UNIVERSITY, Al Ain (AE)

(72) Inventors: VYSHAK SURESHKUMAR, AL AIN (AE); KAILAS VYSHAK, AL AIN (AE)

(21) Appl. No.: 17/360,519

(22) Filed: Jun. 28, 2021

(51) Int. Cl. A61B 5/01; A61B 5/00; A62B 18/08; A62B 23/02; G09F 9/33; G09F 9/30; G09G 3/32; G09G 5/34; G09G 3/00

(52) U.S. Cl. CPC A61B 5/01; A61B 5/742; A61B 5/6803; A62B 18/08; A62B 23/025; A62B 18/084; G09F 9/33; G09F 9/301; G09G 3/32; G09G 5/34; G09G 3/035; A61B 2562/0271; A61B 2562/0257; H01L 27/156

(57) ABSTRACT

The face mask with temperature display is a filtering face mask with an integrated temperature display for displaying a measured temperature of the wearer. The face mask includes a filtering sheet having opposed front and rear faces, and at least one strap attached to the filtering sheet for securing the filtering sheet over a portion of a wearer's face in a manner similar to that of a conventional filtering face mask. A visual display is secured to the front face of the filtering sheet and a temperature sensor is secured to the rear face of the filtering sheet. The temperature sensor is in communication with the visual display, such that the visual display can display the measured body temperature of the wearer.

### BACKGROUND

### 1. Field

The disclosure of the present patent application relates to personal protective equipment, and particularly to a face mask with temperature display for displaying a measured temperature of the wearer on the face mask.

### 2. Description of the Related Art

In order to minimize the spread of infectious diseases, one preventative measure is maintaining a distance from anyone who is suspected of being infected. In business and social settings, a quick test of potential infection is measuring temperature, where fever may be an indication of infection. Unfortunately, it is time-consuming and laborious to use a conventional thermometer to measure the temperature of each person one comes into contact with.

Social distancing is another preventative measure used to minimize the spread of infectious diseases. Social distancing is a set of non-pharmaceutical interventions or measures intended to prevent the spread of a contagious disease by maintaining a physical distance between people and reducing the number of times people come into close contact with each other. Social distancing typically involves keeping a certain distance from others and avoiding gathering together in large groups. By minimizing the probability that a given uninfected person will come into physical contact with an infected person, disease transmission can be reduced, resulting in fewer deaths. However, even for those who are well intentioned, determining if they are, for example, six feet from another person may be difficult, particularly when such distance is usually performed by visual estimation alone. In addition to the inherent inaccuracy of measurement among the populace, there are many people who simply forget or ignore social distancing guidelines, thus putting others at risk. Thus, a face mask with a temperature display solving the aforementioned problems is desired.

### SUMMARY

The face mask with a temperature display is a filtering face mask with a temperature display for displaying a measured temperature of the wearer on the face mask. The face mask with a temperature display includes a filtering sheet having opposed front and rear faces, and at least one strap secured to the filtering sheet for securing the filtering sheet over a portion of a wearer's face in a manner similar to that of a conventional filtering face mask. It should be understood that the filtering sheet and the at least one strap may have any suitable composition and configuration, such as those commonly associated with surgical masks, cloth face masks, N95 face masks, and the like.

A visual display is attached to the front face of the filtering sheet and a temperature sensor is secured to the rear face of the filtering sheet. The temperature sensor is in communication with the visual display, such that the visual display can display the measured temperature of the wearer. A proximity sensor may also be attached to the front face of the filtering sheet. The proximity sensor is in communication with the visual display, such that the visual display can display warning messages to one or more people who are detected within a predetermined proximity to the wearer.

These and other features of the present subject matter will become readily apparent upon further review of the following specification.

### BRIEF DESCRIPTION OF THE DRAWINGS

FIG. 1 is an environmental, perspective view of a face mask with temperature display.

FIG. 2 is a front view of the face mask with temperature display.

FIG. 3 is a rear view of the face mask with temperature display.

FIG. 4 is a block diagram showing system components of the face mask with temperature display.

FIG. 5 is a rear view of a visual display of the face mask with temperature display.

Similar reference characters denote corresponding features consistently throughout the attached drawings.

### DETAILED DESCRIPTION OF THE PREFERRED EMBODIMENTS

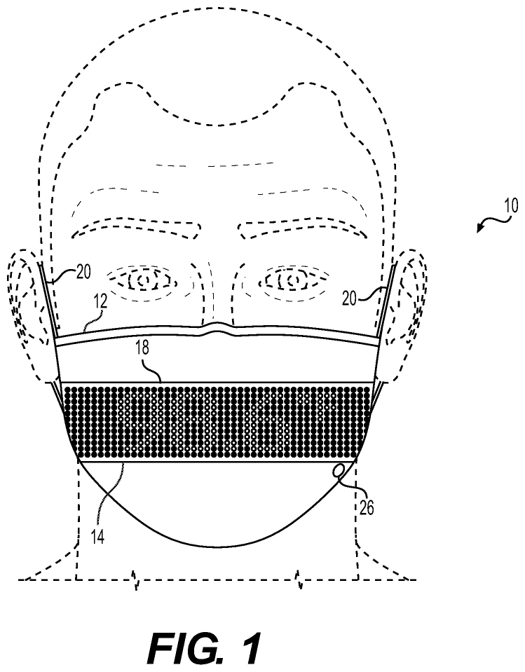

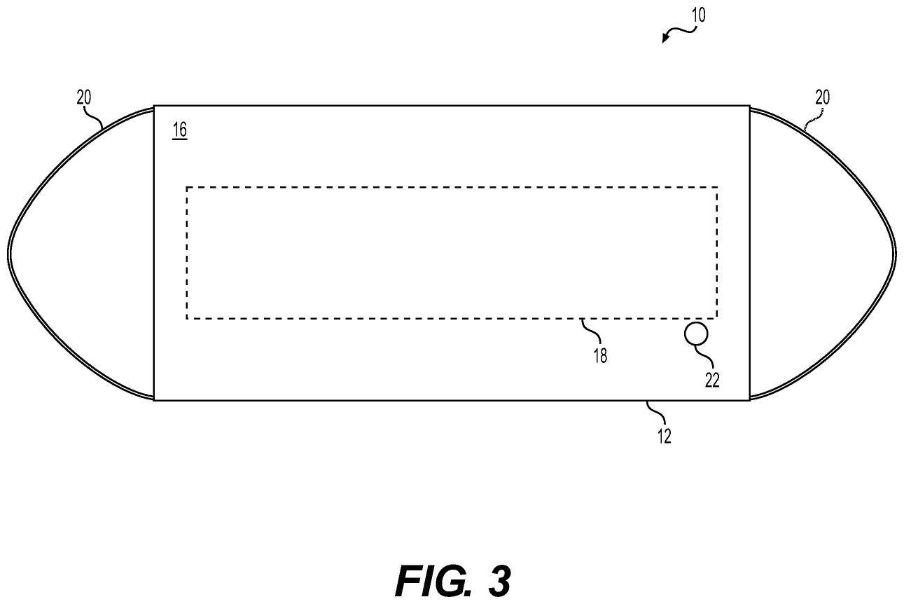

The face mask with temperature display, designated generally as 10 in the drawings, is a filtering face mask with an integrated temperature display for displaying a measured body temperature of the wearer. As shown in FIGS. 1-3, the face mask 10 includes a filtering sheet 12 having opposed front and rear faces 14, 16, respectively, and at least one strap 20 secured to the filtering sheet 12 for securing the filtering sheet 12 over a portion of a wearer's face in a manner similar to that of a conventional filtering face mask. It should be understood that the filtering sheet 12 and the at least one strap 20 may have any suitable composition and configuration, such as those commonly associated with surgical masks, cloth face masks, N95 face masks, and the like. It should be further understood that the overall configuration and relative dimensions of the filtering sheet 12 and straps 20 are shown in FIGS. 1-3 for exemplary purposes only and may vary, depending on the desired type, shape and size of the mask. Additionally, FIGS. 1-3 show the straps 20 as two conventional ear loops, although it should be understood that this is shown for exemplary purposes only, and that any suitable type of strap or straps may be utilized.

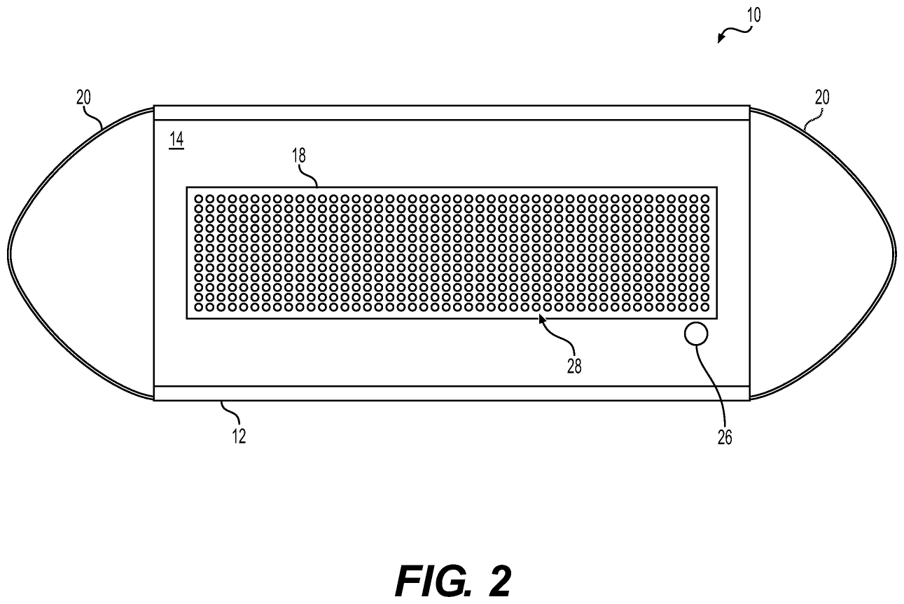

A visual display 18 is secured to the front face 14 of the filtering sheet 12 and a temperature sensor 22 is secured to the rear face 16 of the filtering sheet 12. The temperature sensor 22 is in communication with the visual display 18, such that the visual display 18 can display the measured temperature of the wearer. In the example shown in FIG. 1, the temperature is displayed in Fahrenheit, although it should be understood that the temperature may also be displayed in Celsius, depending on the user's preference. Additionally, it should be understood that the visual display 18 may be any suitable type of visual display, such as a flexible LED display, an array of light emitting diodes, a liquid crystal display, or the like. In the non-limiting example of FIGS. 1 and 2, visual display 18 is shown as a flexible array of light emitting diodes 28. Flexible arrays of light emitting diodes are well known, and it should be understood that any suitable type of flexible array of light emitting diodes, or any other suitable type of display panel, may be used. A suitable face mask with a programmable and removable flexible LED display is marketed by Lumen Couture, www.lumencouture.com/buy-led-light-up-dresses/shop-tech-warning-face-mask/. Also, it should be understood that the location of the temperature sensor 22 is shown for exemplary purposes only, and that the temperature sensor 22 may be positioned at any suitable location for measuring the body temperature of the wearer.

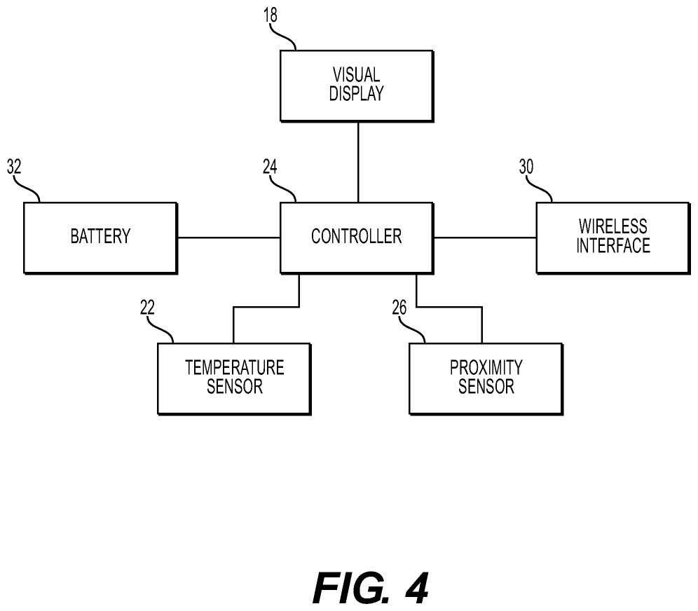

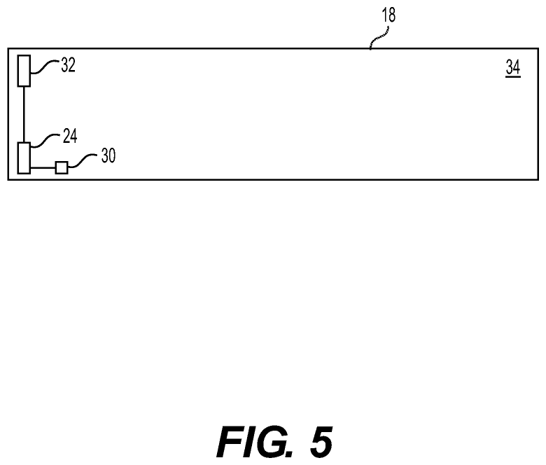

It should be further understood that the temperature sensor 22 may be any suitable type of temperature sensor. For example, a suitable sensor is the LM35 Precision Centigrade Temperature Sensor, manufactured by Texas Instruments®. Alternatively, an infrared temperature sensor may be used to measure the wearer's body temperature. Additionally, as shown in FIG. 5, a controller 24 may be secured to the face mask 10, e.g., to a rear face 34 of the visual display 18. As shown in FIGS. 4 and 5, the controller 24 may be in communication with a wireless interface 30, such as a Bluetooth® interface module or the like, for wirelessly communicating with the temperature sensor 22. Alternatively, the controller 24 may communicate with temperature sensor 22 through a wired connection. Power may be supplied by a rechargeable battery 32 or the like. It should be understood that the controller 24, the wireless interface 30, and the battery 32 are shown attached to the rear face 34 of the visual display 18 for exemplary purposes only, and that each component may be mounted on the face mask 10 at any suitable location. It should be further understood that the controller 24 may be any suitable type of controller, processor, programmable logic controller, or the like. For example, the controller 24 may be an Arduino microcontroller, such as an Arduino Uno microcontroller, manufactured by Arduino®. The microcontroller 24 may be programmed to display the wearer's body temperature on the flexible LED display 18 at intervals in response to sensor input from the temperature sensor 22, as well as a social distancing warning to stay back six feet (or two meters) from the wearer, scrolling the messages across the display 18 as needed.

Additionally, as shown in FIGS. 1 and 2, a proximity sensor 26 may also be attached to the front face 14 of the filtering sheet 12. The proximity sensor 26 is also in communication with the visual display 18 through controller 24, such that the visual display 18 can display warning messages to one or more people who are detected within a predetermined proximity to the wearer. For example, the controller 24 may be programmed with a proximity threshold of 2 m. Thus, if proximity sensor 26 detects one or more people within 2 m of the wearer, the controller 24 can actuate the visual display 18 to display a warning message, such as, “Please keep a distance of 2 m,”, either in response to input from the proximity sensor 26, or at timed intervals, as desired by the user. Proximity sensors are well known, and it should be understood that the proximity sensor 26 may be any suitable type of proximity sensor, such as an optical proximity sensor, an acoustic proximity sensor, or the like. Similar to the temperature sensor 22, a wireless interface 30 associated with the controller 24 may be used to wirelessly communicate with the proximity sensor 26. Alternatively, the controller 24 may communicate with the proximity sensor 26 through a wired connection. Further, it should be understood that the location of the proximity sensor 26 is shown for exemplary purposes only, and that the proximity sensor 26 may be positioned at any suitable location for measuring the proximity of other people in the vicinity of the wearer.

It is to be understood that the face mask with temperature display is not limited to the specific embodiments described above, but encompasses any and all embodiments within the scope of the generic language of the following claims enabled by the embodiments described herein, or otherwise shown in the drawings or described above in terms sufficient to enable one of ordinary skill in the art to make and use the claimed subject matter.

1. A face mask with temperature display, comprising:
a filtering sheet having opposed front and rear faces;
at least one strap attached to the filtering sheet, the at least one strap being adapted for securing the filtering sheet over a portion of a wearer's face;
a visual display attached to the front face of the filtering sheet; and
a temperature sensor attached to the rear face of the filtering sheet, the temperature sensor being in communication with the visual display, the visual display being adapted to display a measured body temperature of the wearer; wherein the visual display and the temperature sensor are distinct devices and are attached to opposing faces of the filtering sheet.

2. The face mask with temperature display as recited in claim 1, wherein the visual display comprises a flexible display.

3. The face mask with temperature display as recited in claim 2, wherein the visual display comprises a flexible light emitting diodes (LED) display.

4. The face mask with temperature display as recited in claim 1, further comprising a proximity sensor attached to the front face of the filtering sheet, the proximity sensor being in communication with the visual display, the visual display being further adapted to display warning messages to one or more people detected within a predetermined proximity to the wearer.

5. A face mask with temperature display, comprising:
a filtering sheet having opposed front and rear faces;
at least one strap attached to the filtering sheet, the at least one strap being adapted for securing the filtering sheet over a portion of a wearer's face;
a visual display attached to the front face of the filtering sheet;
a temperature sensor attached to the rear face of the filtering sheet, the temperature sensor being in communication with the visual display, the visual display being adapted to display a measured temperature of the wearer; and
a proximity sensor attached to the front face of the filtering sheet, the proximity sensor being in communication with the visual display, the visual display being further adapted to display warning messages to one or more people detected within a predetermined proximity to the wearer; wherein the visual display and the temperature sensor are distinct devices and are attached to opposing faces of the filtering sheet.

6. The face mask with temperature display as recited in claim 5, wherein the visual display comprises a flexible display.

7. The face mask with temperature display as recited in claim 6, wherein the visual display comprises a flexible light emitting diodes (LED) display.

8. A face mask with temperature display, comprising:
a face mask having a central filtering portion having a front face and a pair of ear loops extending from the central portion for securing the face mask to a wearer with the central portion covering the wearers nose and mouth;
a programmable flexible light emitting diode (LED) display removably attached to the face mask and extending across the central portion of the face mask in order to be visible to persons looking at the wearer, the display being capable of showing messages scrolling across the display;
a temperature sensor attached to the face mask and configured to contact the face of the wearer, the temperature sensor being configured for emitting a signal proportional to the body temperature of the wearer derived from contact with the wearer's face; and
a microcontroller electrically connected to the flexible LED display and to the temperature sensor, the microcontroller being programmed to actuate the flexible LED display to scroll a message corresponding to the wearer's body temperature across the display in response to receiving the signal proportional to the body temperature of the wearer from the temperature sensor; wherein the flexible LED display and the temperature sensor are distinct devices and are attached to opposing faces of the central filtering portion.

9. The face mask with temperature display according to claim 8, wherein the message corresponding to the wearer's body temperature displays the wearer's body temperature in degrees Celsius.

10. The face mask with temperature display according to claim 8, wherein the message corresponding to the wearer's body temperature displays the wearer's body temperature in degrees Fahrenheit.

11. The face mask with temperature display according to claim 8, wherein said microcontroller is programmed to also scroll a social distancing warning message across the display in addition to scrolling the message corresponding to the wearer's body temperature across the flexible LED display.
